(19) United States
(12) Patent Application Publication
(10) Pub. No.: US 2004/0176470 A1
(43) Pub. Date: Sep. 9, 2004

(54) Method for treatment and chemoprevention of prostate cancer

(76) Inventors: Mitchell S. Steiner, Germantown, TN (US); Sharan Raghow, Collierville, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/747,685

(22) Filed: Dec. 30, 2003

Related U.S. Application Data
(63) Continuation-in-part of application No. 10/611,056, filed on Jul. 2, 2003.
Application No. 10/611,056 is a continuation-in-part of application No. 09/707,766, filed on Nov. 8, 2000, now Pat. No. 6,632,447.
Application No. 09/707,766 is a continuation-in-part of application No. 09/531,472, filed on Mar. 20, 2000, now Pat. No. 6,413,533.
Application No. 09/531,472 is a continuation-in-part of application No. 09/436,208, filed on Nov. 8, 1999, now abandoned.
Application No. 09/436,208 is a continuation-in-part of application No. 09/306,958, filed on May 7, 1999, now Pat. No. 6,265,448.
(60) Provisional application No. 60/084,602, filed on May 7, 1998.

(51) Int. Cl. A61K 31/137

(52) U.S. Cl. 514/651

(57) ABSTRACT

This invention relates to methods of treating a subject with pre-malignant lesions of prostate cancer; and methods of suppressing, inhibiting or reducing the incidence of pre-malignant lesions of prostate cancer.

### CROSS REFERENCE TO RELATED APPLICATION

This application is a Continuation-in-Part application of U.S. Ser. No. 10/611,056, filed Jul. 2, 2003; which is a Continuation-in-Part application of U.S. Ser. No. 09/707,766, filed Nov. 8, 2000, now U.S. Pat. No. 6,632,447, issued Nov. 14, 2003; which is a Continuation-in-Part application of U.S. Ser. No. 09/531,472, filed Mar. 20, 2000, now U.S. Pat. No. 6,413,533, issued Jul. 2, 2002; which is a Continuation-in-Part application of U.S. Ser. No. 09/436,208, filed Nov. 8, 1999, which is a Continuation-in-Part application of U.S. Ser. No. 09/306,958, filed May 7, 1999, now U.S. Pat. No. 6,265,448, which claims priority of U.S. Provisional Application No. 60/084,602, filed May 7, 1998, which are hereby incorporated by reference in their entirety.

### FIELD OF INVENTION

This invention relates to the treatment and prevention of prostate cancer. More particularly, the present invention provides 1) methods of preventing prostate carcinogenesis in a subject; 2) methods of preventing the recurrence of, suppressing, inhibiting or reducing the incidence of prostate carcinogenesis in a subject; 3) methods of treating a subject with prostate cancer; 4) methods of suppressing, inhibiting or reducing the incidence of prostate cancer in a subject; 5) methods of treating a subject with pre-malignant lesions of prostate cancer; and/or 6) methods of suppressing, inhibiting or reducing the incidence of pre-malignant lesions of prostate cancer in a subject by administering to the subject a compound of formula (I) and/or SERM and/or an analog or metabolite thereof, its N-oxide, ester, pharmaceutically acceptable salt, hydrate, or any combination thereof as described herein.

### BACKGROUND OF THE INVENTION

Prostate cancer is one of the most frequently occurring cancers among men in the United States, with hundreds of thousands of new cases diagnosed each year. Unfortunately, over sixty percent of newly diagnosed cases of prostate cancer are found to be pathologically advanced, with no cure and a dismal prognosis. One approach to this problem is to find prostate cancer earlier through screening programs and thereby reduce the number of advanced prostate cancer patients. Another strategy, however, is to develop drugs to prevent prostate cancer. One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, yet the frequency of clinically aggressive cancer is markedly different. This suggests that environmental factors may play a role in activating latent prostate cancer. Thus, the development of chemoprevention strategies against prostate cancer may have the greatest overall impact both medically and economically against prostate cancer.

Because of the high incidence and mortality of prostate cancer, it is imperative to develop chemoprevention strategies against this devastating disease. Understanding those factors that contribute to prostate carcinogenesis, including the initiation, promotion, and progression of prostate cancer, will provide molecular mechanistic clues as to appropriate points of intervention to prevent or halt the carcinogenic process. New innovative approaches are urgently needed at both the basic science and clinical levels to decrease the incidence of prostate cancer as well as to halt or cause the regression of latent prostate cancer. As the frequency of prostate cancer escalates dramatically at the same ages at which men are confronted by other competing causes of mortality, simply slowing the progression of prostate adenocarcinoma may be both a more suitable and a cost effective health strategy.

Various approaches have been taken to the chemoprevention of prostate cancer. Greenwald, “Expanding Horizons in Breast and Prostate Cancer Prevention and Early Detection” in J. Cancer Education, 1993, Vol. 8, No. 2, pages 91-107, discusses the testing of 5a-reductase inhibitors such as finasteride for the prevention of prostate cancer. Brawley et al., “Chemoprevention of Prostate Cancer” in Urology, 1994, Vol. 43, No. 5, also mentions 5a-reductase inhibitors as well as difluoromethylornithine and retinoids as potential chemopreventive agents.

Kelloff et al., “Introductory Remarks: Development of Chemopreventive Agents for Prostate Cancer” in Journal of Cellular Biochemistry, 1992, Supplement 16H: 1-8, describes National Cancer Institute preclinical studies of seven agents: alltrans-N-(4-hydroxyphenyl)retinamide, difluoromethylornithine, dehydroepiandrosterone, liarozole, lovastatin, oltipraz, and finasteride.

Lucia et al., “Chemopreventive Activity of Tamoxifen, N-(4-Hydroxyphenyl) retinamide, and the Vitamin D Analogue Ro24-553 1 for Androgen-promoted Carcinomas of the Rat Seminal Vesicle and Prostate” in Cancer Research, 1995, Vol. 55, pages 5621-5627, reports chemoprevention of prostate carcinomas in Lobund-Wistar rats by tamoxifen, an estrogen response modifier.

As discussed in Potter et al., “A mechanistic hypothesis for DNA adduct formation by tamoxifen following hepatic oxidative metabolism” in Carcinogenesis, 1994, Vol. 15, No. 3, pages 439-442, tamoxifen causes liver carcinogenicity in rats, which is attributed to the formation of covalent DNA adducts. This reference also reports that the tamoxifen analogue toremifene, which showed a much lower level of hepatic DNA adduct formation than tamoxifen, is non-carcinogenic.

Toremifene is an example of a triphenylalkene compound described in U.S. Pat. Nos. 4,696,949 and 5,491,173 to Toivola et al., the disclosures of which are incorporated herein by reference. The parenteral and topical administration to mammalian subjects of formulations containing toremifene are described in U.S. Pat. No. 5,571,534 to Jalonen et al. and in U.S. Pat. No. 5,605,700 to DeGregorio et al., the disclosures of which are incorporated herein by reference.

Toremifene-containing formulations for reversing the multidrug resistance to cancer cells to a cytotoxic drug are described in U.S. Pat. No. 4,990,538 to Harris et al., the disclosure of which is incorporated herein by reference.

U.S. Pat. Nos. 5,595,722 and 5,599,844 to Grainger et al., the disclosures of which are incorporated herein by reference, describe methods for identifying agents that increase TGFP levels and for orally administering formulations containing TGFP activators and TGFP production stimulators to prevent or treat conditions characterized by abnormal proliferation of smooth muscle cells, for example, vascular trauma. Disclosed agents for increasing TGFP levels include tamoxifen and its analogue toremifene.

U.S. Pat. Nos. 5,629,007 and 5,635,197 to Audia et al., the disclosures of which are incorporated herein by reference, describe a method of preventing the development of prostatic cancer in a patient at risk of developing such cancer, for example, a patient having benign prostatic hyperplasia, by administering to the patient an octahydrobenzo [f} quinolin-3-one compound.

U.S. Pat. No. 5,595,985 to Labrie, the disclosure of which is incorporated herein by reference, also describes a method for treating benign prostatic hyperplasia using a combination of a 5a-reductase inhibitor and a compound that binds and blocks access to androgen receptors. One example of a compound that blocks androgen receptors is flutamide.

U.S. Pat. Nos. 4,329,364 and 4,474,813 to Neri et al., the disclosures of which are incorporated herein by reference, describe pharmaceutical compositions comprising flutamide for delaying and/or preventing the onset of prostate carcinoma. The preparation can be in the form of a capsule, tablet, suppository, or elixir.

Despite these developments, there is a continuing need for agents and methods effective for preventing prostate cancer. The present invention is directed to satisfying this need.

### SUMMARY OF THE INVENTION

This invention relates to the treatment and chemoprevention of prostate cancer. More particularly, the present invention provides 1) methods of preventing prostate carcinogenesis in a subject; 2) methods of preventing the recurrence of, suppressing, inhibiting or reducing the incidence of prostate carcinogenesis in a subject; 3) methods of treating a subject with prostate cancer; 4) methods of suppressing, inhibiting or reducing the incidence of prostate cancer in a subject; 5) methods of treating a subject with pre-malignant lesions of prostate cancer; and 6) methods of suppressing, inhibiting or reducing the incidence of pre-malignant lesions of prostate cancer in a subject. The methods of the present invention comprise administering to the subject a compound of formula (I) and/or SERM and/or an analog or metabolite thereof or its pharmaceutically acceptable salts, esters, N-oxide, or mixtures thereof and/or an analog or metabolite thereof, as described herein.

The present invention provides a safe and effective method for suppressing or inhibiting prostate cancer, e.g. latent prostate cancer, and is particularly useful for treating subjects having an elevated risk of developing prostate cancer, for example those having benign prostatic hyperplasia, prostate intraepithelial neoplasia (PIN), or an abnormally high level of circulating prostate specific antibody (PSA) or having a family history of prostate cancer.

This invention provides a method of administering to a subject an effective dose of an antiestrogen that does not cause the formation of DNA adducts to treat, prevent, prevent recurrence of, suppress, and/or inhibit prostate cancer and to treat, prevent, prevent recurrence of, suppress, and/or inhibit pre-malignant lesions of prostate cancer. This invention provides a method of administering to a subject an effective dose of a SERM to treat, prevent, prevent recurrence of, suppress, and/or inhibit prostate cancer and to treat, prevent, prevent recurrence of, suppress, and/or inhibit pre-malignant lesions of prostate cancer. In one embodiment, the antiestrogen is a compound of formula (I). In another embodiment, the methods of the present invention comprise administering pharmaceutically acceptable salts, esters, N-oxide, or mixtures thereof of the compound of formula (I). In another embodiment, the methods of the present invention comprise administering an analog and/or metabolite of the compound of formula (I).

Thus, in one embodiment, this invention provides a method of preventing prostate carcinogenesis in a subject, comprising the step of administering to the subject a pharmaceutical composition comprising from about 20 mg to about 60 mg of a compound represented by the structure of formula (I) and/or an analog or metabolite thereof, its N-oxide, ester, pharmaceutically acceptable salt, hydrate, or any combination thereof:

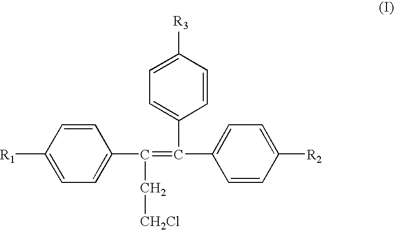

wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms.

In another embodiment, this invention provides a method of preventing the recurrence of, suppressing, inhibiting or reducing the incidence of prostate carcinogenesis, or increasing the survival rate of a subject having prostate cancer, said method comprising the step of administering to said subject a pharmaceutical composition comprising from about 20 mg to about 60 mg of a compound represented by the structure of formula (I), and/or an analog or metabolite thereof, its N-oxide, ester, pharmaceutically acceptable salt, hydrate, or any combination thereof.

In one embodiment, the subject has an elevated risk of prostate cancer. In another embodiment, the subject has benign prostatic hyperplasia, prostatic intraepithelial neoplasia(PIN), or an abnormally high level of circulating prostate specific antibody (PSA).

In another embodiment, the present invention provides a method of treating a subject with prostate cancer, comprising the step of administering to the subject a pharmaceutical composition comprising from about 20 mg to about 60 mg of a compound represented by the structure of formula (I) and/or an analog or metabolite thereof, its N-oxide, ester, pharmaceutically acceptable salt, hydrate, or any combination thereof.

In another embodiment, the present invention provides a method of suppressing, inhibiting or reducing the incidence of prostate cancer in a subject, comprising the step of administering to the subject a pharmaceutical composition comprising from about 20 mg to about 60 mg of a compound represented by the structure of formula (I) and/or an analog or metabolite thereof, its N-oxide, ester, pharmaceutically acceptable salt, hydrate, or any combination thereof.

In one embodiment, the prostate cancer is latent prostate cancer. In another embodiment, the subject has a precancerous precursor of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

In another embodiment, the present invention provides a method of suppressing, inhibiting or reducing the incidence of pre-malignant lesions of prostate cancer in a subject, comprising the step of administering to the subject a pharmaceutical composition comprising from about 20 mg to about 60 mg of a compound represented by the structure of formula (I) and/or an analog or metabolite thereof, its N-oxide, ester, pharmaceutically acceptable salt, hydrate, or any combination thereof.

In another embodiment, the present invention provides a method of treating a subject with pre-malignant lesions of prostate cancer, comprising the step of administering to the subject a pharmaceutical composition comprising from about 20 mg to about 60 mg of a compound represented by the structure of formula (I) and/or an analog or metabolite thereof, its N-oxide, ester, pharmaceutically acceptable salt, hydrate, or any combination thereof.

In one embodiment, the pre-malignant lesion is a precancerous precursor of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

In one embodiment, the compound of formula (I) is toremifene and/or an analog or metabolite thereof, its N-oxide, ester, pharmaceutically acceptable salt, hydrate, or any combination thereof.

In one embodiment, the pharmaceutical composition comprises about 20 mg of the compound of formula (I). In another embodiment, the pharmaceutical composition comprises about 40 mg of the compound of formula (I). In another embodiment, the pharmaceutical composition comprises about 60 mg of the compound of formula (I).

The present invention provides a safe and effective method for 1) treating a mammalian subject with prostate cancer; 2) suppressing, inhibiting or reducing the incidence of prostate cancer in a mammalian subject; 3) reducing the risk of developing prostate cancer in a mammalian subject; 4) treating precancerous precursors of prostate adenocarcinoma lesions in a mammalian subject; 5) suppressing or inhibiting precancerous precursors of prostate adenocarcinoma lesions in a mammalian subject; and 6) reducing the amount of precancerous precursors of prostate adenocarcinoma lesions in a mammalian subject and is particularly useful for treating subjects having an elevated risk of developing prostate cancer, for example, those having benign prostatic hyperplasia, prostate intraepithelial neoplasia (PIN), or an abnormally high level of circulating prostate specific antibody (PSA) or having a family history of prostate cancer.

In another embodiment, this invention provides a method of preventing the recurrence of, suppressing, inhibiting or reducing the incidence of prostate carcinogenesis, or increasing the survival rate of a subject having prostate cancer, said method comprising the step of administering to said subject a pharmaceutical composition comprising from about 20 mg to about 60 mg of a SERM, and/or an analog or metabolite thereof, its N-oxide, ester, pharmaceutically acceptable salt, hydrate, or any combination thereof.

In one embodiment, the subject has an elevated risk of prostate cancer. In another embodiment, the subject has benign prostatic hyperplasia, prostatic intraepithelial neoplasia(PIN), or an abnormally high level of circulating prostate specific antibody (PSA).

In another embodiment, the present invention provides a method of treating a subject with prostate cancer, comprising the step of administering to the subject a pharmaceutical composition comprising from about 20 mg to about 60 mg of a SERM and/or an analog or metabolite thereof, its N-oxide, ester, pharmaceutically acceptable salt, hydrate, or any combination thereof.

In another embodiment, the present invention provides a method of suppressing, inhibiting or reducing the incidence of prostate cancer in a subject, comprising the step of administering to the subject a pharmaceutical composition comprising from about 20 mg to about 60 mg of a SERM and/or an analog or metabolite thereof, its N-oxide, ester, pharmaceutically acceptable salt, hydrate, or any combination thereof.

In one embodiment, the prostate cancer is latent prostate cancer. In another embodiment, the subject has a precancerous precursor of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

In another embodiment, the present invention provides a method of suppressing, inhibiting or reducing the incidence of pre-malignant lesions of prostate cancer in a subject, comprising the step of administering to the subject a pharmaceutical composition comprising from about 20 mg to about 60 mg of a SERM and/or an analog or metabolite thereof, its N-oxide, ester, pharmaceutically acceptable salt, hydrate, or any combination thereof.

In another embodiment, the present invention provides a method of treating a subject with pre-malignant lesions of prostate cancer, comprising the step of administering to the subject a pharmaceutical composition comprising from about 20 mg to about 60 mg of a SERM and/or an analog or metabolite thereof, its N-oxide, ester, pharmaceutically acceptable salt, hydrate, or any combination thereof.

In one embodiment, the pre-malignant lesion is a precancerous precursor of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

In one embodiment, the pharmaceutical composition comprises about 20 mg of the SERM. In another embodiment, the pharmaceutical composition comprises about 40 mg of the SERM. In another embodiment, the pharmaceutical composition comprises about 60 mg of the SERM.

### BRIEF DESCRIPTION OF THE DRAWINGS

FIG. 1: A graph illustrating the chemopreventive effects of toremifene in the TRAMP model.

FIGS. 2A-2C: H&E sections illustrating ventral prostate cells in normal mice and prostate carcinoma in TRAMP mice included in the study.

FIG. 3: Effect of toremifene on ventral prostate development in the TRAMP mice.

FIG. 4: Effect of toremifene on tumor occurrence in the TRAMP mice.

FIG. 5: Effect of toremifene on tumor development in the TRAMP model.

FIGS. 6A-6B: Comparison of placebo vs. toremifene effects on tumor growth.

### DETAILED DESCRIPTION OF THE INVENTION

This invention relates to the treatment and chemoprevention of prostate cancer. More particularly, the present invention provides 1) methods of preventing prostate carcinogenesis in a subject; 2) methods of preventing the recurrence of, suppressing, inhibiting or reducing the incidence of prostate carcinogenesis in a subject; 3) methods of treating a subject with prostate cancer; 4) methods of suppressing, inhibiting or reducing the incidence of prostate cancer in a subject; 5) methods of treating a subject with pre-malignant lesions of prostate cancer; and 6) methods of suppressing, inhibiting or reducing the incidence of pre-malignant lesions of prostate cancer in a subject, by administering to the subject an antiestrogen that does not cause the formation of DNA adducts, for example a compound of formula (I), and/or an analog or metabolite thereof, and/or a SERM or an analog and/or metabolite thereof, its N-oxide, ester, pharmaceutically acceptable salt, hydrate, or any combination thereof.

In another embodiment, the present invention provides a method of suppressing, inhibiting or reducing the incidence of pre-malignant lesions of prostate cancer in a subject, comprising the step of administering to the subject a pharmaceutical composition comprising from about 20 mg to about 60 mg of a SERM and/or an analog or metabolite thereof, its N-oxide, ester, pharmaceutically acceptable salt, hydrate, or any combination thereof.

In another embodiment, the present invention provides a method of treating a subject with pre-malignant lesions of prostate cancer, comprising the step of administering to the subject a pharmaceutical composition comprising from about 20 mg to about 60 mg of a SERM and/or an analog or metabolite thereof, its N-oxide, ester, pharmaceutically acceptable salt, hydrate, or any combination thereof.

In one embodiment, the pre-malignant lesion is a precancerous precursor of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

In one embodiment, the pharmaceutical composition comprises about 20 mg of the SERM. In another embodiment, the pharmaceutical composition comprises about 40 mg of the SERM. In another embodiment, the pharmaceutical composition comprises about 60 mg of the SERM.

This invention provides a method of administering to a subject an effective dose of an antiestrogen that does not cause the formation of DNA adducts to treat, prevent, prevent recurrence of, suppress, and/or inhibit prostate cancer and to treat, prevent, prevent recurrence of, suppress and/or inhibit pre-malignant lesions of prostate cancer. In one embodiment, the antiestrogen is a compound of formula (I). In another embodiment, the methods of the present invention comprise administering a pharmaceutically acceptable salt, ester, N-oxide, hydrate or mixtures thereof of the compound of formula (I). In another embodiment, the methods of the present invention comprise administering an analog and/or metabolite of the compound of formula (I). A composition and/or a pharmaceutical composition can also comprise the compound of formula (I).

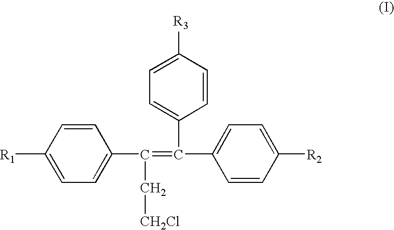

wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms.

Thus, in one embodiment, this invention provides a method of preventing prostate carcinogenesis in a subject, comprising the step of administering to the subject a pharmaceutical composition comprising from about 20 mg to about 60 mg of an antiestrogen, for example a compound represented by the structure of formula (I), its N-oxide, ester, pharmaceutically acceptable salt, hydrate, or any combination thereof and/or an analog or metabolite thereof.

In another embodiment, this invention provides a method of preventing the recurrence of, suppressing, inhibiting or reducing the incidence of prostate carcinogenesis, or increasing the survival rate of a subject having prostate cancer, said method comprising the step of administering to said subject a pharmaceutical composition comprising from about 20 mg to about 60 mg of an antiestrogen, for example a compound represented by the structure of formula (I), its N-oxide, ester, pharmaceutically acceptable salt, hydrate, or any combination thereof, and/or an analog or metabolite thereof.

In one embodiment, the subject has an elevated risk of prostate cancer. In another embodiment, the subject has benign prostatic hyperplasia, prostatic intraepithelial neoplasia(PIN), or an abnormally high level of circulating prostate specific antibody (PSA).

In another embodiment, the present invention provides a method of treating a subject with prostate cancer, comprising the step of administering to the subject a pharmaceutical composition comprising from about 20 mg to about 60 mg of an antiestrogen, for example a compound represented by the structure of formula (I), its N-oxide, ester, pharmaceutically acceptable salt, hydrate, or any combination thereof.

In another embodiment, the present invention provides a method of suppressing, inhibiting or reducing the incidence of prostate cancer in a subject, comprising the step of administering to the subject a pharmaceutical composition comprising from about 20 mg to about 60 mg of an antiestrogen, for example a compound represented by the structure of formula (I), its N-oxide, ester, pharmaceutically acceptable salt, hydrate, or any combination thereof, and/or an analog or metabolite thereof.

In one embodiment, the prostate cancer is latent prostate cancer. In another embodiment, the subject has a precancerous precursors of prostate adenocarcinoma. In another embodiment, the precancerous precursors of prostate adenocarcinoma is prostate intraepithelial neoplasia (PIN). In another embodiment, the prostate intraepithelial neoplasia is high grade prostate intraepithelial neoplasia (HGPIN).

In another embodiment, the present invention provides a method of suppressing, inhibiting or reducing the incidence of pre-malignant lesions of prostate cancer in a subject, comprising the step of administering to the subject a pharmaceutical composition comprising from about 20 mg to about 60 mg of an antiestrogen, for example a compound represented by the structure of formula (I), its N-oxide, ester, pharmaceutically acceptable salt, hydrate, or any combination thereof, and/or an analog or metabolite thereof.

In another embodiment, the present invention provides a method of treating a subject with pre-malignant lesions of prostate cancer, comprising the step of administering to the subject a pharmaceutical composition comprising from about 20 mg to about 60 mg of an antiestrogen, for example a compound represented by the structure of formula (I), its N-oxide, ester, pharmaceutically acceptable salt, hydrate, or any combination thereof, and/or an analog or metabolite thereof.

In one embodiment, the pre-malignant lesion is a precancerous precursor of prostate adenocarcinoma. In another embodiment, the precancerous precursor of prostate adenocarcinoma is prostate intraepithelial neoplasia (PIN). In another embodiment, the prostate intraepithelial neoplasia is high grade prostate intraepithelial neoplasia (HGPIN).

In one embodiment, the methods of the present invention comprise administering an analog of the antiestrogen. In another embodiment, the methods of the present invention comprise administering a derivative of the antiestrogen. In another embodiment, the methods of the present invention comprise administering an isomer of the antiestrogen. In another embodiment, the methods of the present invention comprise administering a metabolite of the antiestrogen. In another embodiment, the methods of the present invention comprise administering a pharmaceutically acceptable salt of the antiestrogen. In another embodiment, the methods of the present invention comprise administering an ester of the antiestrogen. In another embodiment, the methods of the present invention comprise administering an N-oxide of the antiestrogen. In another embodiment, the methods of the present invention comprise administering any of a combination of an analog, derivative, isomer, metabolite, pharmaceutically acceptable salt, ester, or N-oxide of the antiestrogen. In one embodiment, the antiestrogen is a chemopreventive agent useful for inhibiting, suppressing, and/or inhibiting prostate carcinogenesis or pre-malignant lesions of prostate cancer.

In one embodiment, the methods of the present invention comprise administering an analog of the compound of formula (I). In another embodiment, the methods of the present invention comprise administering a derivative of the compound of formula (I). In another embodiment, the methods of the present invention comprise administering an isomer of the compound of formula (I). In another embodiment, the methods of the present invention comprise administering a metabolite of the compound of formula (I). In another embodiment, the methods of the present invention comprise administering a pharmaceutically acceptable salt of the compound of formula (I). In another embodiment, the methods of the present invention comprise administering an ester of the compound of formula (I). In another embodiment, the methods of the present invention comprise administering an N-oxide of the compound of formula (I). In another embodiment, the methods of the present invention comprise administering any of a combination of an analog, derivative, isomer, metabolite, pharmaceutically acceptable salt, ester, or N-oxide of the compound of formula (I). In one embodiment, the compound of formula (I) is a chemopreventive agent useful for inhibiting, suppressing, and/or inhibiting prostate carcinogenesis or pre-malignant lesions of prostate cancer.

In one embodiment, the antiestrogen is toremifene, its N-oxide, ester, pharmaceutically acceptable salt, hydrate, its analog or metabolite, or any mixtures thereof. Toremifene is chemically named 4-chloro-1,2-diphenyl-1-[4-[2-(N,N-dimethylamino)ethoxy]phenyl]-1-butene, i.e. the compound of formula (I), wherein R₁ and R₂ are each H and R₄ and R₅ are each methyl. Toremifene has been shown safe and effective as an anti-tumor compound and exhibits hormonal effects as an estrogenic or as an anti-estrogenic agent, depending on the dosage used.

On administration, toremifene has several metabolites that are also biologically active, which are well known to those skilled in the art, which are also useful for treating, preventing, preventing recurrence of, suppressing, and/or inhibiting prostate cancer and for treating, preventing, preventing recurrence of, suppressing, and/or inhibiting pre-malignant lesions of prostate cancer. These analogs and/or metabolites include but are not limited to 4-chloro-1,2-diphenyl-1-[4-[2-(N-methylamino)ethoxy]phenyl]-1-butene; 4-chloro-1,2-diphenyl-1-[4-[2-(N,N-diethylamino)ethoxy]phenyl]-1-butene; 4-chloro-1,2-diphenyl-1-[4(aminoethoxy)]-1-butene; 4-chloro-1-(4-hydroxyphenyl)-1-[4-[2-(N,N-dimethylamino)ethoxy]phenyl]-2-phenyl-1-butene; 4-chloro-1-(4-hydroxyphenyl)-1-[4-[2-(N-methylamino)ethoxy]phenyl]-2-phenyl-1-butene; and 4-chloro-1,2-bis(4-hydroxyphenyl)-1-[4-[2-(N,N-dimethylamino)ethoxy]phenyl ]-1-butene.

In the experiments conducted herein, in both animal and human studies the antiestrogen was shown to prevent prostate cancer. The prostates were actually dissected and evaluated both histologically and by wholemount analysis. Also, toremifene was tested for the prevention of prostate cancer by treating LNCaP xenografts in nude mice. As is shown, the data is quite dramatic. Not only has an antiestrogen such as toremifene-inhibited growth, but toremifene was actually able to produce regression of the tumors. Further, human studies conducted with the antiestrogen high grade PIN (HGPIN), which has been established and time tested as a precursor lesion for human prostate cancer (latent prostate cancer), have shown regression, thus demonstrating that the antiestrogen toremifene is a prostate chemopreventive agent.

This invention also provides a method of administering to a subject an effective dose of an antiestrogen that does not cause the formation of DNA adducts to treat, prevent, prevent recurrence of, suppress, and/or inhibit prostate cancer and to treat, prevent, prevent recurrence of, suppress, and/or inhibit pre-malignant lesions of prostate cancer.

Embodiments of antiestrogens that act as prostate chemopreventive agents and/or are useful for treating prostate cancer or pre-malignant lesions of prostate cancer include but are not limited to: toremifene and analogs or synthetics thereof, selective estrogen receptor modulators (SERMS), triphenylethylenes which include droloxifen, idoxifene, tamoxifen, (2)4-OH-tamoxifene; arzoxifene; chromans such as levomeloxifene and centchroman; benzothiophenes such as raloxifene and LY 353381; naphthalens such as CP336,156; phytoestrogens such as isoflavanoids including daidzein, genistein, yenoestrogens; coumestrol: zearalenone; daidzein; apigenin; waempferol; phioretin; biochanin A; naringenin; formononetin; ipriflavone; quercetin; chrysin; flavonoids; flavones, isoflavones, flavanones, and chalcones); coumestans; mycoestrogens; resorcydic acid factone: nafoxideneand equol, and lignan including enterodiol and enterolactone; and other compounds that are known in the art as follows: ICI 164,384, ICI 182,780; TAT-59, EM-652 (SCG 57068), EM-800 (SCH57050), EM-139, EM-651, EM-776, and peptide antagonist of human estrogen receptors. In another embodiment the chemopreventive agent is faslodex or its analog, derivative, isomer, metabolite, pharmaceutically acceptable salt, ester, or N-oxide, or mixtures thereof.

This invention provides the use of a pharmaceutical composition for preventing prostate cancer, preventing the recurrence of, suppressing or inhibiting prostate carcinogenesis, or increasing the survival rate of a subject having prostate cancer, comprising an antiestrogen that does not cause the formation of DNA adducts and a suitable diluent. The antiestrogens include the antiestrogens provided above.

In one embodiment the compound of the present invention is an antiestrogen, its analog, derivative, isomer, and metabolite thereof. In another embodiment the antiestrogen is a tri-phenylalkane or, its analog, derivative, isomer, and metabolite thereof. In another embodiment the antiestrogen is a dihydronapthalene, its analog, derivative, isomer, and metabolite thereof. In another embodiment the antiestrogen is a benzothiopheneor its analog, derivative, isomer, metabolite thereof. In another embodiment the antiestrogen is a selective estrogen receptor modulator (SERM) and its analog, derivative, isomer, and metabolite thereof. In another embodiment, the antiestrogen is a non; DNA adduct forming antiestrogen and its analog, derivative, isomer, and metaboitte thereof. In one embodiment the antiestrogen is tamoxifen. In another embodiment the antiestrogen is faslodex. In another embodiment the antiestrogen raloxifene.

As contemplated herein, this invention encompasses the use of analogs, derivatives, isomers, metabolites, pharmaceutically acceptable salts, esters, or N-oxides, or any mixtures thereof of the compounds described herein.

As defined herein, the term “isomer” includes, but is not limited to, optical isomers and analogs, structural isomers and analogs, conformational isomers and analogs, and the like.

In one embodiment, this invention encompasses the use of various optical isomers of the compounds described herein. It will be appreciated by those skilled in the art that the antiestrogens may contain at least one chiral center. Accordingly, the compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof. In one embodiment, the compounds are the pure (R)-isomers. In another embodiment, the compounds are the pure (S)-isomers. In another embodiment, the compounds are a mixture of the (R) and the (S) isomers. In another embodiment, the compounds are a racemic mixture comprising equal amounts of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention encompasses pure (Z)- and (E)-isomers of the compounds and mixtures thereof, as well as pure (RR,SS)- and (RS,SR)-enantiomer couples and mixtures thereof.

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the compounds of formula (I). The term “derivatives” includes but is not limited to ether derivatives, acid derivatives, amide derivatives, ester derivatives, and the like. In addition, this invention further includes hydrates of the compounds of the present invention. The term “hydrate” includes but is not limited to hemihydrate, monohydrate, dihydrate, trihydrate, and the like.

This invention further includes metabolites of the compounds of formula (I). The term “metabolite” means any substance produced from another substance by metabolism or a metabolic process.

The antiestrogen agents, for example the compounds of formula (I) can be prepared according to procedures described in the previously cited U.S. Pat. Nos. 4,696,949 and 5,491,173 to Toivola et al., the contents of which are incorporated by reference in their entirety herein.

As used herein, “pharmaceutical composition” means therapeutically effective amounts of the antiestrogen together with suitable diluents, preservatives, solubilizers, emulsifiers, adjuvants, and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral. In one embodiment, the pharmaceutical composition is administered parenterally, paracancerally, transmucosally, transdermally, intramuscularly, intravenously, intradermally, subcutaneously, intraperitonealy, intraventricularly, intracranially, or intratumorally.

The dosage of the compound may be in the range of 5-80 mg/day. In another embodiment the dosage is in the range of 35-66 mg/day. In another embodiment the dosage is in the range of 40-60 mg/day. In another embodiment the dosage is in a range of 45-60 mg/day. In another embodiment the dosage is in the range of 15-25 mg/day. In another embodiment the dosage is in the range of 55-65 mg/day. In another embodiment the dosage is in the range of 45-60 mg/day. In another embodiment the dosage is in the range of 60-80 mg/day. In another embodiment the dosage is 20 mg/day. In another embodiment the dosage is 40 mg/day. In another embodiment the dosage is 60 mg/day. In another embodiment the dosage is 80 mg/day.

In one embodiment, the dosage is 20 mg/day. In another embodiment, the dosage is 40 mg/day. In another embodiment, the dosage is 60 mg/day. In one embodiment, the dosage is 20 mg/day and the antiestrogen is toremifene. In another embodiment, the dosage is 40 mg/day and the antiestrogen is toremifene. In another embodiment, the dosage is 60 mg/day and the antiestrogen is toremifene. In another embodiment, the dosage is 80 mg/day and the antiestrogen is toremifene.

In one embodiment, the pharmaceutical composition comprises about 20 mg to about 60 mg of the antiestrogen. In another embodiment, the pharmaceutical composition comprises about 20 mg of the antiestrogen. In another embodiment, the pharmaceutical composition comprises about 40 mg of the antiestrogen. In another embodiment, the pharmaceutical composition comprises about 60 mg of the antiestrogen. In another embodiment, the pharmaceutical composition comprises about 20 mg to about 30 mg of the antiestrogen. In another embodiment, the pharmaceutical composition comprises about 30 mg to about 40 mg of the antiestrogen. In another embodiment, the pharmaceutical composition comprises about 40 mg to about 50 mg of the antiestrogen. In another embodiment, the pharmaceutical composition comprises about 50 mg to about 60 mg of the antiestrogen. In another embodiment, the pharmaceutical composition comprises about 60 mg to about 70 mg of the antiestrogen. In another embodiment, the pharmaceutical composition comprises about 70 mg to about 80 mg of the antiestrogen. In one embodiment the antiestrogen is the compound of formula I. In another embodiment the antiestrogen is a SERM.

In one embodiment, the pharmaceutical composition comprises about 20 mg to about 60 mg of toremifene. In another embodiment, the pharmaceutical composition comprises about 20 mg of toremifene. In another embodiment, the pharmaceutical composition comprises about 40 mg of toremifene. In another embodiment, the pharmaceutical composition comprises about 60 mg of toremifene. In another embodiment, the pharmaceutical composition comprises about 20 mg to about 30 mg of toremifene. In another embodiment, the pharmaceutical composition comprises about 30 mg to about 40 mg of toremifene. In another embodiment, the pharmaceutical composition comprises about 40 mg to about 50 mg of toremifene. In another embodiment, the pharmaceutical composition comprises about 50 mg to about 60 mg of toremifene.

Further, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, or emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, and suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases, and the like.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands, or antigens or coupled to ligands of tissue-specific receptors. Other embodiments of the compositions of the invention incorporate particulate forms, protective coatings, protease inhibitors, or permeation enhancers for various routes of administration, including parenteral, pulmonary, nasal, and oral. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone, or polyproline, are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the antiestrogen agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol.2, pp.115-138 (1984). Preferably, a controlled release device is introduced into a subject in proximity of the site of inappropriate immune activation or a tumor. Other controlled release systems are discussed in the review by Langer (Science 249:1527-1533 (1990).

The method of the present invention for treating and/or preventing prostate carcinogenesis involves administering to a mammalian subject a pharmaceutical composition comprising the antiestrogen or a metabolite or salt thereof. The pharmaceutical composition can comprise the compounds of the present invention alone or can further include a pharmaceutically acceptable carrier and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof The pharmaceutical composition containing the active agent can be administered to a subject by, for example, subcutaneous implantation of a pellet; in a further embodiment, the pellet provides for controlled release of active agent over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository. The pharmaceutical composition can also be a parenteral formulation; in one embodiment, the formulation comprises a liposome that includes a complex of a active agents such as, for example, toremifene and a cyclodextrir compound, as described in the previously cited U.S. Pat. No. 5,571,534 to Jalonen et al.

The pharmaceutical compositions of the invention can be prepared by known dissolving, mixing, granulating, or tablet-forming processes. For oral administration, the compounds of the present invention or their physiologically tolerated derivatives such as salts, esters, N-oxides, and the like are mixed with additives customary for this purpose, such as vehicles, stabilizers, or inert diluents, and converted by customary methods into a suitable form for administration, such as tablets, coated tablets, hard or soft gelatin capsules, aqueous, alcoholic, or oily solutions. Examples of suitable inert vehicles are conventional tablet bases such as lactose, sucrose, or cornstarch in combination with binders like acacia, cornstarch, gelatin, or with disintegrating agents such as cornstarch, potato starch, alginic acid, or with a lubricant such as stearic acid or magnesium stearate. Examples of suitable oily vehicles or solvents are vegetable or animal oils such as sunflower oil or fish-liver oil. Preparations can be effected as dry or as wet granules. For parenteral administration (subcutaneous, intravenous, intraarterial, or intramuscular injection), the compounds of the present invention or their physiologically tolerated derivatives such as salts, esters, N-oxides, and the like are converted into a solution, suspension, or emulsion, if desired, with the substances customary and suitable for this purpose, for example, solubilizers or other auxiliaries. Examples are: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose, and related sugar solutions, and glycols such as propylene glycols or polyethylene glycol are preferred liquid carriers, particularly for injectable solutions.

The preparation of pharmaceutical compositions that contain an active component is well understood in the art. Typically, such compositions are prepared as an aerosol of the polypeptide delivered to the nasopharynx or as injectables, either as liquid solutions or suspensions, although solid forms suitable for solution in, or suspension in, liquid prior to injection can also be prepared. The preparation can also be emulsified. The active therapeutic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, or pH buffering agents, which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule) and are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the active agents or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp.353-365 (1989); Lopez-Berestein, ibid., pp.317-327; see generally ibid).

The compositions and pharmaceutical compositions of the present invention are particularly useful for treating a subject having an elevated risk of developing prostate cancer. High-risk subjects include, for example, those having benign prostatic hyperplasia, prostatic intraepithelial neoplasia (PIN), an abnormally high level of circulating prostate specific antibody (PSA), or having a family history of prostate cancer.

Further, the antiestrogen compounds of the present invention may be administered in combination with other cytokines or growth factors that include but are not limited to: IFN, alpha or beta; interleukin (IL) 1, IL-2, IL-4, IL-6, IL-7, IL-12, tumor necrosis factor (TNF) □, TNF-□, granulocyte colony stimulating factor (G-CSF), granulocyte/macrophage CSF (GM-CSF); accessory molecules, including members of the integrin superfamily and members of the Ig superfamily such as, but not limited to, LFA-1, LFA-3, CD22, and B7-1, B7-2, and ICAM-1 T cell costimulatory molecules.

Furthermore, treatment with the antiestrogen compounds of the present invention may precede or follow a DNA-damaging agent treatment by intervals ranging from minutes to weeks. Protocols and methods are known to those skilled in the art. DNA-damaging agents or factors are known to those skilled in the art and refer to any chemical compound or treatment method that induces DNA damage when applied to a cell. Such agents and factors include radiation and waves that induce DNA damage, such as gamma-irradiation, X-rays, UV-irradiation, microwaves, electronic emissions, and the like. A variety of chemical compounds, also described as “chemotherapeutic agents”, function to induce DNA damage, all of which are intended to be of use in the combined treatment methods disclosed herein. Chemotherapeutic agents contemplated to be of use include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA-damaging agents, whether radiation-based or actual compounds, such as the use of X-rays with cisplatin or the use of cisplatin with etoposide.

In another embodiment, one may irradiate the localized tumor site with DNA-damaging radiation such as X-rays, UV-light, gamma-rays, or even microwaves. Alternatively, the tumor cells may be contacted with the DNA-damaging agent by administering to the subject a therapeutically effective amount of a pharmaceutical composition comprising a DNA-damaging compound, such as adriamycin, 5-fluorouracil, etoposide, camptothecin, actinomycin-D, mitomycin C, or more preferably, cisplatin. Agents that damage DNA also include compounds that interfere with DNA replication, mitosis, and chromosomal segregation. Such chemotherapeutic compounds include adriamycin, also known as doxorubicin, etoposide, verapamil, podophyllotoxin, and the like.

Other factors that cause DNA damage and have been used extensively include what are commonly known as gamma-rays, X-rays, and/or the directed delivery of radioisotopes to tumor cells. Other forms of DNA-damaging factors are also contemplated, such as microwaves and UV-irradiation. It is most likely that all of these factors effect a broad range of damage to DNA, on the precursors of DNA, the replication and repair of DNA, and the assembly and maintenance of chromosomes.

As can be readily appreciated by one of ordinary skill in the art, the methods and pharmaceutical compositions of the present invention are particularly suited to administration to a mammal, preferably a human subject.

Intermediate endpoint biomarkers are measurable biologic alterations in tissue that occur between the initiation of and the development of frank neoplasia. A biomarker is validated if the final endpoint, cancer incidence, is also reduced by the putative compounds of the present invention. Intermediate biomarkers in cancer may be classified into the following groups: histologic, proliferation, differentiation, and biochemical markers. In any chemoprevention strategy, the availability of histologically recognizable and accepted precancerous lesions constitutes an important starting point. For the prostate, a histological marker is a precancerous precursor of prostatic adenocarcinoma, of which prostatic intraepithelial neoplasia (PIN) is an example. PIN appears as an abnormal proliferation within the prostatic ducts of premalignant foci of cellular dysplasia and carcinoma in situ without stromal invasion. PIN and histological prostate cancer are morphometrically and phenotypically similar. Thus, the development of high-grade PIN represents an important step in the progression pathway whereby the normal prostate develops PIN, histological prostate cancer, invasive clinical prostate cancer, and metastases.

Prostate intraepithelial neoplasia has been shown to be a precancerous lesion, or precursor of prostatic adenocarcinoma. Prostate intraepithelial neoplasia is the abnormal proliferation within the prostatic ducts of premalignant foci of cellular dysplasia and carcinoma in situ without stromal invasion. Prostate intraepithelial neoplasia is the most accurate and reliable marker of prostate carcinogenesis and may be used as an acceptable endpoint in prostate chemoprevention trials. Prostate intraepithelial neoplasia has a high predictive value as a marker for adenocarcinoma, and its identification warrants repeat biopsy for concurrent or subsequent invasive carcinoma. Most studies suggest that most patients with prostate intraepithelial neoplasia will develop carcinoma within 10 years. Interestingly, prostate intraepithelial neoplasia does not contribute to serum PSA, which is not surprising, since, unlike prostate cancer, prostate intraepithelial neoplasia has not yet invaded the vasculature of the prostate to leak PSA into the blood stream. Thus, prostate intraepithelial neoplasia may precede even prostate-cancer related serum PSA elevations.

The following examples are presented in order to more fully illustrate the preferred embodiments of the invention. They should in no way be construed, however, as limiting the broad scope of the invention.

### EXPERIMENTAL DETAILS SECTION

### EXAMPLE 1

### Transgenic Adenocarcinoma Mouse Prostate

The study of prostate cancer chemoprevention has been hindered by the lack of appropriate animal models. The recent development of the transgenic adenocarcinoma mouse prostate (TRAMP) model enables the study of chemoprevention. In the TRAMP model, which is described in Greenberg et al., “A Prostate cancer in a transgenic mouse”, Proc. Natl Acad. Sci. USA, 1995, Vol. 92, pages 3439-3443, the PB-SV40 large T antigen (PB-Tag) transgene is expressed specifically in the epithelial cells of the murine prostate. As a result, this model has several advantages over currently existing models: 1) mice develop progressive forms of prostatic epithelial hyperplasia as early as 10 weeks and invasive adenocarcinoma around 18 weeks of age; 2) the metastatic spread of prostate cancer pattern mimics human prostate cancer with the common sites of metastases being lymph node, lung, kidney, adrenal gland, and bone; 3) the development as well as the progression of prostate cancer can be followed within a relatively short period of 10-30 weeks; 4) the tumors arise with 100% frequency; and 5) the animals may be screened for the presence of the prostate cancer transgene prior to the onset of clinical prostate cancer to directly test treatment with chemopreventive agents that may alter prostate carcinogenesis.

The TRAMP transgenic mouse model is an excellent in vivo model to determine the mechanisms of initiation and promotion of prostate cancer and to test the effectiveness of potential chemopreventive agents. These mice progressively develop prostatic epithelial hyperplasia, PIN, and then prostate cancer within a short period (<17 weeks).

Chemopreventive treatment of hybrid TRAMP mice is initiated 30 days postnatally, using chemopreventive agents at a level of about 0.5-50 mg/kg of subject weight/day, preferably about 6-30 mg/kg of subject weight/day. The chemopreventive agents are conveniently processed into 21-day and 90-day pellets (prepared by Innovative Research of America, Sarasota, Fla.) and delivered as subcutaneous implants. Control animals receive placebo implants. In each drug treatment group, animals are sacrificed at 5, 7, 10, 15, 20, 25, 30, 40, and 50 weeks of age until the development of a palpable tumor. Blood is collected and pooled per treatment time point to evaluate changes in serum testosterone and estradiol. Prostatic tissues are harvested for morphometric, histologic, and molecular studies.

The following test procedures are employed:

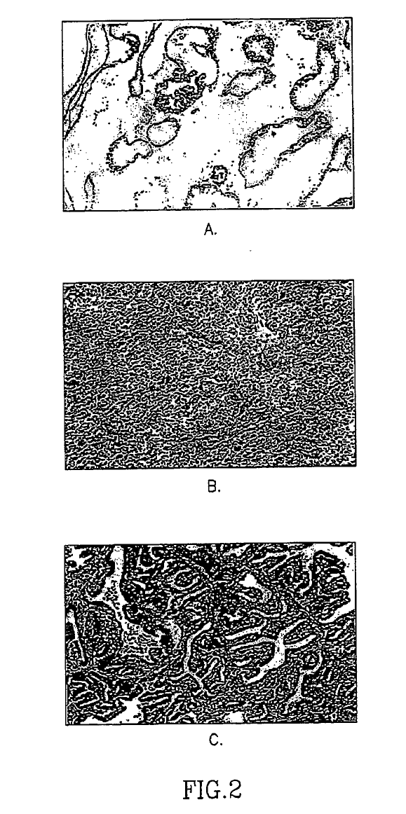

1) Prostate wholemount analysis is serially performed to detect changes in prostate ductal morphology over time with and without treatment; examples are shown in FIG. 2. Tissue sections are evaluated histologically by H&E and Masson-trichome standard staining. The emergence of PIN is assessed and graded (I-mild to III-severe).

2) Serum estradiol and total testosterone levels are measured (RIA) for each age interval to assess any changes in these hormones as a result of chemopreventive agents.

### EXAMPLE 2

### Immunohistochemistry Data Analysis

Microscopy images of each tissue section are evaluated using computer-assisted (Mac 9500-132 computer and monitor) image quantitation (NIH-Image 1.6 PPC) using Kodak DCS 460 camera on Nikon Microphot-FX microscope and quantitated using a color-assisted quantitative system image analysis (IPLab Spectrum 3.1, Scanalytics, Inc., VA) that distinguishes color differences of stained tissue sections. Thresholds are set to identify various tissue components of the prostate. The area pixel densities corresponding to each of these tissue components are calculated for each full screen of the color monitor. A total of 5 screens per prostate section are averaged. Immunohistochemical images can be digitalized and quantitated to enable statistical evaluation by determination of sample correlation coefficients and probability (2-tailed).

### EXAMPLE 3

### Study of Chemopreventive Activity

A study was undertaken to test the efficacy of chemopreventive agents in TRAMP transgenic animals (PBTag×FVBwt)(provided by Dr. Norman Greenberg, Baylor College of Medicine, TX). These mice showed preliminary signs of cancer as early as 10 weeks. The TRAMP transgenic male litters were screened for the Large Tag transgene, and the positive males were used in the study. The antiestrogen toremifene, which was to be tested for its possible chemopreventive effects, was incorporated in customized pellets (Innovative Research of America, Sarasota, Fla.), and chemopreventive treatment of mice was initiated postnatally at 30 days (average mouse weight 14 g). Four groups of 10-12 animals each received subcutaneous implantations of 90 day-release toremifene-containing pellets. The diffusible drug dosage, adjusted for growth related changes in weight, was designed to deliver either a low dose (6 mg/kg) or a high dose (30 mg/kg) of toremifene. Control animals (n=10) received placebo implants. The efficacy of the treatment was measured by the absence of palpable tumor formation. The murine prostate tumors were harvested and evaluated by molecular and histological techniques.

Using the TRAMP transgenic model of prostate cancer, in which every animal that inherits the prostate cancer gene develops prostate cancer, it was demonstrated that toremifene both increases the latency and decreases the incidence of prostate cancer.

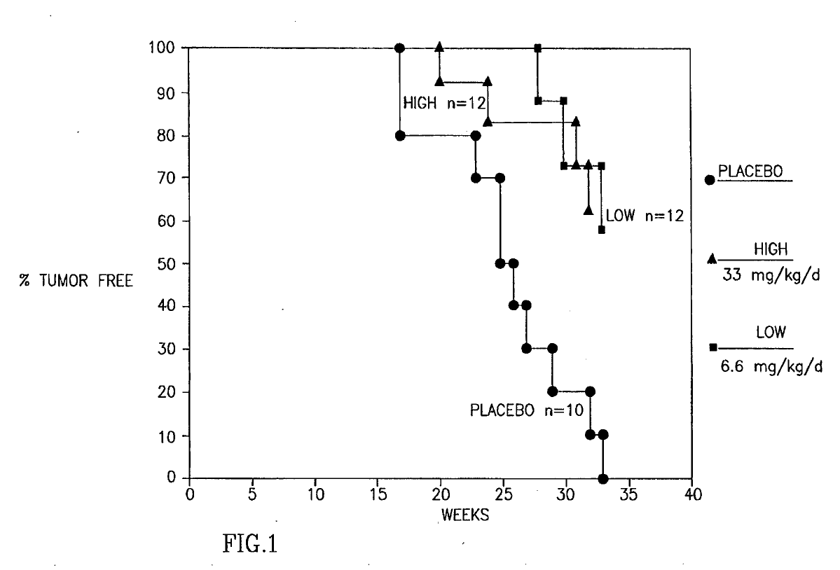

As shown in FIG. 1, the low and high doses of toremifene were both effective. Tumor formation in the TRAMP mouse ventral prostate was noted at week 17 for the placebo group (n=10), at week 19 for the high dose toremifene-treated group(n=12), and at week 28 for the low dose toremifene-treated group (n=12). Thus, 5 treatments by toremifene substantially increased the latency period by up to 11 weeks for the development of cancer in the ventral prostate of TRAMP mice.

Since the toremifene-treated animals did not reach the 50% tumor development point during the period of the study, the time by which 25% of the animals had tumors was compared among groups. Tumors were palpable in 25% of 10 the animals by week 23 in the placebo group and by 30-31 weeks in the high and low toremifene groups, a delay of 7-8 weeks. Both low toremifene and high toremifene vs placebo were significant by log rank and Wilcoxon statistical analysis, as shown in Table 1 below.

TABLE 1
Statistical Analysis

| | Log-Rank P | Wilcoxon p |
|---|---|---|
| Low toremifene vs placebo | 0.0003* | 0.0004* |
| High toremifene vs placebo | 0.0017* | 0.0071* |

*significance P < 0.05

At week 33, a point when all of the control animals had developed tumors, 72% of the low dose and 60% of the high dose toremifene-treated animals were still tumor-free. Thus, toremifene treatment at both low and high dosages resulted in a greatly decreased incidence of tumors in the ventral prostate of TRAMP mice. These data demonstrated that the incidence of prostate cancer was significantly decreased and the latency period increased.

As already discussed, administering toremifene produces a substantial chemopreventive effect against tumors in the ventral prostate of TRAMP mice. This result is encouraging for a similar beneficial effect on human subjects, whose prostate includes a segment corresponding to the ventral prostate of rodents.

### EXAMPLE 4

### Histological Examination of Prostate Tissue

Tumors from the placebo and high-toremifene treated groups taken at the time of palpation were evaluated histologically. FIG. 2A is an H&E section of the ventral prostate of a 17-week-old normal adult mouse. FIG. 2B, a section of the ventral prostate of a placebo-treated 16-week-old TRAMP mouse, shows that, unlike the normal prostate structure depicted in FIG. 2A, the TRAMP mouse ventral prostate is characterized by sheets of undifferentiated, anaplastic cells with a high mitotic index. In contrast, as shown in FIG. 2C, the prostate of a toremifene-treated 30-week-old TRAMP mouse retains much of the normal glandular architecture and has tumors with a more differentiated structure, the mitotic index being much lower than that for the placebo-treated animal. These results indicate that toremifene, even at low, is able to suppress prostate carcinogenesis in the TRAMP model.

Western blot analysis: Prostate tissues (dorsolateral and ventral lobes) were harvested at 10 weeks of age, snap-frozen in liquid N₂ and stored at −80° C. Tissue lysates were prepared using RIPA buffer (150 mM NaCl, 1% NP40, 0.5% deoxycholate, 0.1% SDS and 50 mM Tris, pH 7.5) containing a cocktail of protease inhibitors (Pefabloc, aprotinin, bestatin, leupeptin, and pepstain) and the phosphatase inhibitor Na₃VO₄ (10 mM). The homogenate was centrifuged at 14,000×g at 4° C. for 10 minutes and lysates were stored at −80° C.

Protein concentrations were determined using the Bradford protein assay (Bio-Rad). Tissue lysates were loaded onto 7.5% polyacrylamide gels, proteins (40 μg/lane) separated by SDS-PAGE, and electrophoretically transferred to nitrocellulose membranes (0.2 μm, Bio-Rad, Hercules, Calif.) in transfer buffer (192 mM glycine, 25 mM Tris-HCl and 20% methanol). TRAMP prostate tumor tissue was used as positive control. Chemiluminescent Cruz Markers (Santa Cruz Biotechnology, Santa Cruz, Calif.) were used as molecular weight standards. Blots were blocked overnight at 4° C. in BLOTTO (6% non-fat dry milk in 1×TBS) and incubated with the large T-antigen primary antibody (Pab 101 mouse monoclonal, 1:200, Santa Cruz Biotechnology) for 2 hours at room temperature. The blots were washed (3×) with TTBS (0.05% Tween 20, 50 mM Tris-Hcl, 200 mM NaCl) and incubated with horseradish peroxidase (HRP)-conjugated secondary antibody (1:5000) for 1 hour at 25° C. Immunoreactive proteins were visualized on autoradiography film using the enhanced chemiluminescence (ECL) system (APB, Piscataway, N.J.). Actin protein expression was used to normalize Tag results. For this purpose, the above membrane was submerged in stripping buffer (100 mM 2-mercaptoethanol, 2% SDS, 62.5 nM Tris-Hcl pH 6.7) and incubated at 50° C. for 30 minutes with occasional agitation. After blocking, the membrane was reprobed with actin primary antibody (1:2500, Chemicon, Temecula, Calif.) followed by (HRP)-conjugated secondary antibody (1:10000). Following ECL detection, band intensities were quantitated using Adobe Photoshop 5.0 Acquisition and ImageQuant Analysis (Molecular Dynamics) systems.

### EXAMPLE 5

### Use of Chemopreventive Efficacy of Toremifene Against Prostate Cancer in the TRAMP Mouse Model

This experiment confirms and demonstrates the chemopreventive efficacy of toremifene. The present study focuses on the histological and molecular changes associated with development of prostate tumor in control animals and the mechanism of toremifene chemopreventive action with TRAMP animals that are bred, screened, and treated with sustained-release drug pellets. At predetermined times, groups of 5 animals were sacrificed and their prostates were removed for analysis. The prostate glands were evaluated for the presence of tumor by histology, wholemount dissections, and large T antigen immunohistochemistry. To date, the placebo and the toremifene treatments have been completed for the 7, 10, 15, and 20 week time-points, and the results are described below.

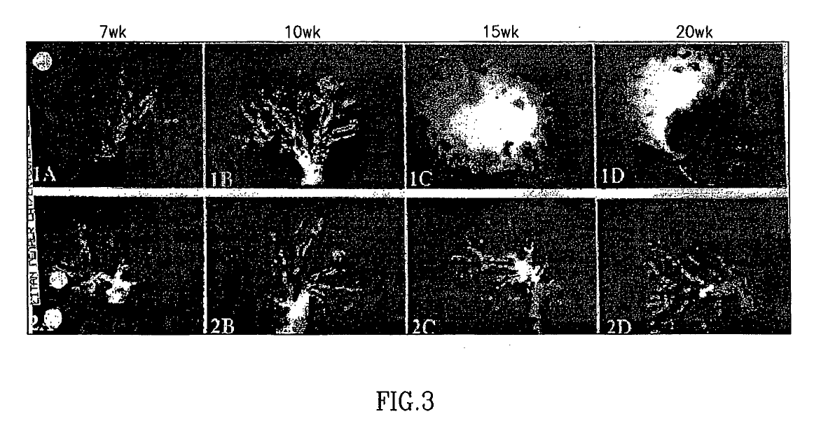

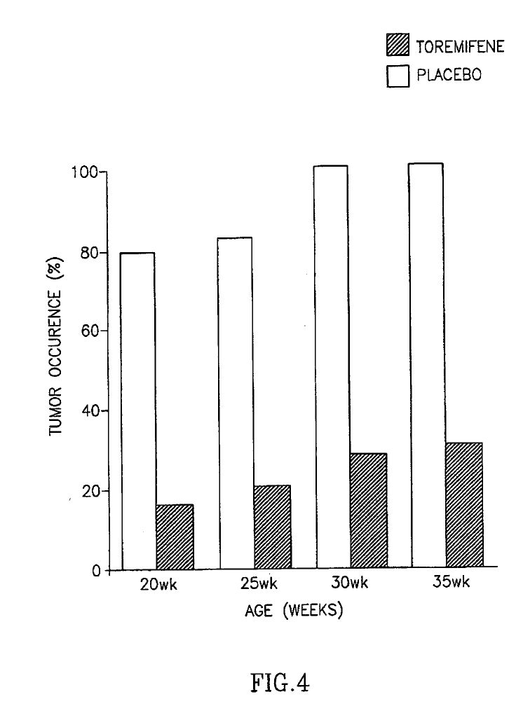

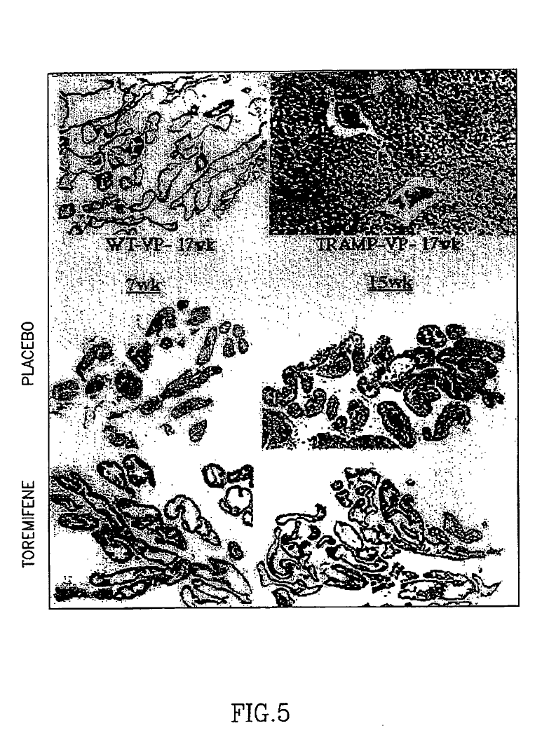

Results: Prostatic wholemounts for 7,10,15, and 20 weeks for the various groups have been completed. Wholemount analysis revealed that placebo-treated mice developed prostate tumors by 15-20 weeks of age, similar to the previous pilot study. Moreover, the toremifene-treated animals had a delay in the occurrence of prostate cancer up to 20 weeks (FIG. 3). By 20 weeks, there is a striking delay in tumor occurrence in the toremifene-treated group up to 35 weeks FIG. 4). These data confirm that even with a more sensitive assessment of tumorigenicity, toremifene exhibited chemopreventive activity. For histological evaluation, tissue samples were fixed, processed, and paraffin embedded. Sections (5 pM thick) were cut and stained by routine H&E method. Toremifene inhibited the ductal development and tissue differentiation (compare the 17-weeks TRAMP mouse prostate tumor vs. wildtype (FIG. 4) and the toremifene-treated prostate histology vs. placebo at 15 weeks (FIG. 5). Qualitatively, immunohistochemistry of placebo- and toremifene-treated tissues showed presence of T-antigen in the ventral prostate. Thus, the chemopreventive activity seen with toremifene does not appear to be due to suppression of the probasin promoter in the TRAMP model.

Conclusions: The ability of toremifene to prevent the occurrence of prostate cancer in the TRAMP model has been confirmed utilizing more sensitive techniques to assess tumor formation. The mechanism of toremifene's chemopreventive effects does not appear to be through loss of the transgene for the Large T-antigen protein.

### EXAMPLE 6

### Toremifene Induces Regression of Established Human Prostate Cancer Tumors in the Nude Mouse Model

Prostate cancer currently remains the most commonly diagnosed cancer in American males. However, questions remain about the etiology and treatment of this disease, especially its advanced forms. Hormone therapy remains the standard method of treatment for recurrent and advanced prostate cancer, despite the common development of hormone refractory disease. Therefore, new approaches to the prevention and treatment of prostate cancer are needed to accommodate the increasing number of men diagnosed with this disease. The experiments and results below demonstrate that toremifene suppresses hormone-sensitive LNCaP tumor growth in athymic nude mice.

Materials and Methods: One million LNCaP cells in Matrigel were subcutaneously injected into each flank of athymic nude mice. A total of 40 mice were injected. After approximately 3-4 weeks, visible tumors developed. After the tumor size was recorded in two dimensions, the mice were divided into placebo and treatment groups based on equivalent tumor burden. A single pellet (placebo versus toremifene 35 mg) was subcutaneously implanted between the scapulae of each mouse. Weekly measurements of the tumor size were recorded. Tumor volume was calculated (tumor volume=0.5 (L+W)×L×W×0.5236, where L=tumor length and W=width). The tumor volume at the time of pellet implantation served as the point of reference for future comparison of that tumor's size variation. The weekly variations of each tumor volume were recorded as percent differentiation from the original measurement at pellet implantation.

Results: Of the 78 tumor injection sites, 55 (70%) resulted in tumors of adequate volume for evaluation. A total of 50 tumors (24 placebo and 26 chemopreventive agent, toremifene-treated animals) were available for evaluation. Mean tumor volumes at the time of pellet implantation were similar for the chemopreventive agent, toremifene, and placebo groups (1.90 mm³ and 1.72 mm³, respectively). Mean tumor volume decreased to 1.68 mm³ in the chemopreventive agent, toremifene group (−0.22 mm³), while mean tumor volume increased to 2.33 mm³ in the placebo group (+0.61 mm³). Mean serum PSA level was higher in the placebo group (3.80 mg/ml) than in the chemopreventive agent, toremifene group (2.80 ng/ml), but this was not statistically significant (p=0.755). Total testosterone serum levels were 2.18 ng/ml for the placebo group (n=17) and 2.96 ng/ml for the chemopreventive agent, toremifene group (n=19).

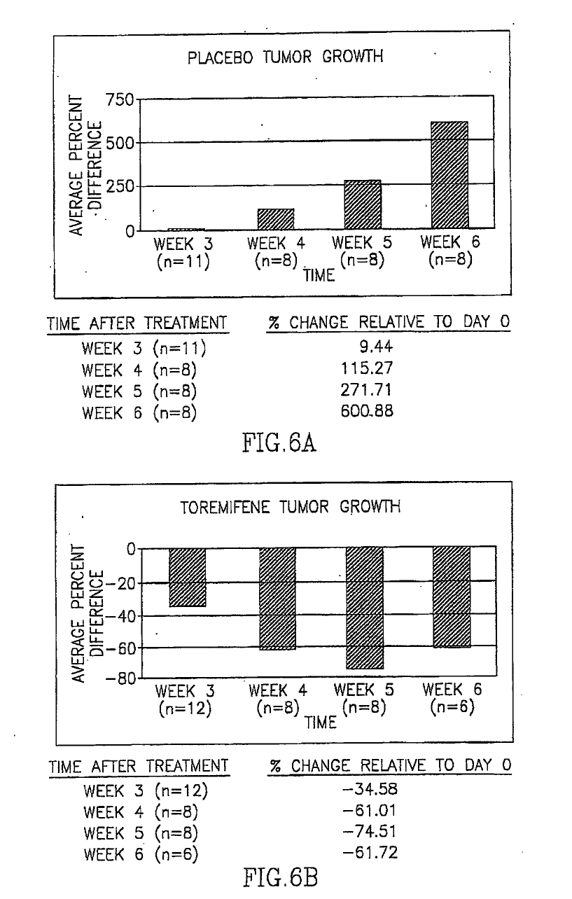

Two mice died soon after pellet implantation due to mortal wounds from other mice. One mouse treated with toremifene was excluded from the study due to excessive tumor hemorrhage and hematoma development. All mice developed visible tumors unilaterally or bilaterally. Each tumor was followed independently for the duration of the study. 24 tumors were treated with placebo and 28 tumors were treated with toremifene. The results are shown in Table 2 and FIGS. 6A and 6B.

TABLE 2

| | | |
|---|---|---|
| | | % Change in volume |
| Week | N= | relative to day 0 of treatment |

PLACEBO GROUP

| | | |
|---|---|---|
| 3 | 11 | 9.44 |
| 4 | 8 | 115.27 |
| 5 | 8 | 271.71 |
| 6 | 8 | 600.88 |

TOREMIFENE GROUP

| | | |
|---|---|---|
| 3 | 11 | −34.58 |
| 4 | 7 | −61.01 |
| 5 | 7 | −74.51 |
| 6 | 5 | −61.72 |

The follow-up interval will be extended on the currently reported population, and data on additional animals are presently being collected.

Conclusion: The chemopreventive agent toremifene inhibits and induces regression of established LNCaP tumors. Although the mechanism by which toremifene exerts this effect is unknown, the ability to produce these effects supports the use of toremifene as a treatment for prostate cancer and to prevent the recurrence of prostate cancer in high-risk patients with established prostate-cancer micrometastases.

### EXAMPLE 7

### The Role of Antiestrogens: Tamoxifen Citrate and Raloxifene (SERMs) and Faslodex (Pure Antiestrogen ICI 182,780) in the Prevention of Prostate Cancer

Experimental design: Chemopreventive treatment of mice is initiated post-natal at 30 days. Three groups of 50 hybrid TRAMP male mice each are treated with either Tamoxifen citrate, Raloxifene (SERMs), or Faslodex (pure antiestrogen ICI 182,780). The drugs are obtained as customized sustained-release pellets (Innovative Research of America, Sarasota, Fla.) and delivered as subcutaneous implants (see preliminary data). Control animals receive placebo implant with no pharmacological activity. Animals (n=10) are sacrificed at periodic intervals, 10, 15, 20, 25 and 30 weeks age, and the efficacy of the treatment leading to either absence of tumor formation or reduction in tumor size, if present, is assessed by comparison with placebo control animals. Blood is collected to evaluate changes in serum androgens and estrogens with each treatment. Prostatic tissues is saved: a) for morphometric studies; b) for histologic studies (the tissue will be fixed in 10% buffered formalin, processed and paraffin embedded); c) for molecular studies (the tissues is frozen in liquid nitrogen and stored at −70° C.). Necropsies and survival data are also recorded.

The results of the experiment reveal the relative chemopreventive efficacy of the various antiestrogens in the delay or prevention of prostate cancer in the TRAMP model. The morphological studies indicate the gross changes, if any, in the development of the prostate size and ductal pattern as a result of each treatment. Paraffinized tissue sections are stained using standard H&E techniques for histological changes such as PIN that will be assessed to monitor the appearance of precancerous lesions as a precursor of prostatic adenocarcinoma. Serum estradiol and total testosterone levels are measured for each age interval to assess any changes in these hormones and whether or not they correlate to changes in PIN. The peptide growth factor levels of TGF, TGF 1, TGF 3, and bFGF are quantitated in prostate samples taken at each interval. Corresponding peptide growth-factor receptors are also assessed for EGFR and TGF RI and RII.

TABLE 3
The effects of Selective Estrogen Receptor Modulators (SERMs) on the
prevention of prostate cancer in the TRAMP model
Animals were sacrificed at 20 weeks, and prostate glands were
evaluated by wholemount analysis and histologically.

| | | 20 wks | 20 wks |
|---|---|---|---|
| SERM | Dose | # tumors | (% tumors) |
| Placebo | — | 5/5 | 100% |
| Toremifene | 20 mg/kg/d | 1/7 | 14.2% |
| Tamoxifen | 20 mg/kg/d | 2/9 | 22% |
| Raloxifene | 20 mg/kg/d | 3/10 | 30% |
| *Faslodex | *10 mg/kg/d | 8/11 | 72% |
| (ICI 128,780) | | | |

*Faslodex is a pure antiestrogen and its relative potency is 2x that of the other SERMs therefore 10 mg/kg/d of Faslodex = 20 mg/kg/d of SERM.

### EXAMPLE 8

### Toremifene Causes Regression of HGPIN in a Phase IIa Prostate-Cancer Chemoprevention Human Clinical Trial

The chemopreventive effects of an antiestrogen, toremifene, against prostate cancer have been reproducibly demonstrated herein in a well-established animal model of spontaneous human prostate cancer. This represents the first compound to demonstrate chemopreventive activity against prostate cancer.

A Phase IIa, open-labeled non-randomized single center study with 21 human subjects was conducted. In this protocol, patients with biopsy proven PIN and who do not have prostate cancer are treated with 60 mg of the chemopreventive agent, toremifene, daily for 4 months. After 4 months, patients are rebiopsied (8 biopsies) and PIN status is reassessed. Twenty-one patients entered the study and sixteen patients have completed the study. The summary of pathologic findings of the prostate biopsies of these 16 patients showed that 12 patients had regression of PIN to benign or atrophic prostate tissue; thus, 12 out of 16 (75%) patients had a complete response. Of the remaining 4 patients, 3 patients had prostate cancer but the amount of PIN was reduced, and 1 patient had stable disease, but the PIN epithelium demonstrated atrophic and degenerative changes.

The pathological evaluation revealed complete resolution of PIN with atrophic changes in the prostatic epithelium. The patients experienced no acute or chronic toxicities while taking toremifene. The serum PSA, serum-free testosterone, serum-total testosterone, and-serum estradiol remained in the normal ranges. Quality of life was unchanged, including no effect on potency and libido. Therefore, these results demonstrate a prostate chemopreventive role for the antiestrogen toremifene.

The results demonstrate that the chemopreventive agents such as toremifene reduce PIN, which thus directly translates to a decrease in the incidence and a prolongation of the latency of prostate cancer and preventing prostate carcinogenesis. Lastly, the chemopreventive agent, toremifene has been found to significantly induce TGFβ synthesis in human stromal fibroblast cells.

It will be appreciated by a person skilled in the art that the present invention is not limited by what has been particularly shown and described hereinabove. Rather, the scope of the invention is defined by the claims that follow:

What is claimed is:

1. A method of suppressing, inhibiting, or reducing the incidence of pre-malignant lesions of prostate cancer in a human comprising the step of administering to the human a pharmaceutical composition comprising a metabolite of a compound represented by the structure of formula (I), its N-oxide, ester, pharmaceutically acceptable salt, hydrate, or any combination thereof: 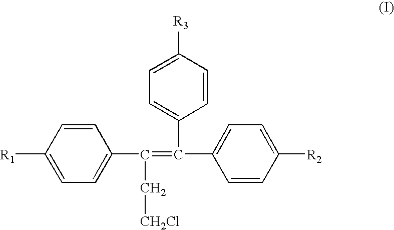
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms.

2. A method of treating a human with pre-malignant lesions of prostate cancer, comprising the step of administering to the human a pharmaceutical composition comprising a metabolite of a compound represented by the structure of formula (I), its N-oxide, ester, pharmaceutically acceptable salt, hydrate, or any combination thereof: 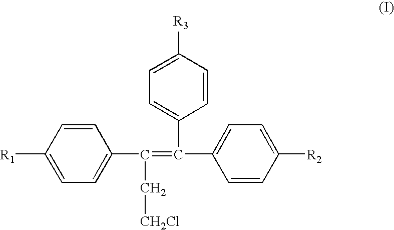
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms.

3. The method according to any of claim 1 or 2, wherein said pharmaceutical composition comprises about 20 mg of the analog or a metabolite of the compound of formula (I).

4. The method according to any of claim 1 or 2, wherein said pharmaceutical composition comprises about 40 mg of the analog or a metabolite of the compound of formula (I).

5. The method according to any of claim 1 or 2, wherein said pharmaceutical composition comprises about 60 mg of the analog or a metabolite of the compound of formula (I).

6. The method according to any of claims 1 or 2, wherein the pre-malignant lesion is a precancerous precursor of prostate adenocarcinoma.

7. The method according to claim 1 or 2, wherein the precancerous precursors of prostate adenocarcinoma is prostate intraepithelial neoplasia (PIN).

8. The method according to claim 7, wherein the prostate intraepithelial neoplasia is high grade prostate intraepithelial neoplasia (HGPIN).

9. The method according to any of claims 1, or 2, wherein said pharmaceutical composition further comprises a pharmaceutically acceptable carrier.

10. The method according to claim 9, wherein said carrier is selected from the group consisting of a gum, a starch, a sugar, a cellulosic material, and mixtures thereof.

11. The method according to any of claims 1, or 2, wherein said administering comprises subcutaneously implanting in said human a pellet containing said pharmaceutical composition.

12. The method according to claim 11, wherein said pellet provides for controlled release of said pharmaceutical composition over a period of time.

13. The method according to any of claims 1, or 2, wherein said administering comprises intravenously, intraarterially, or intramuscularly injecting into said human said pharmaceutical composition in liquid form.

14. The method according to any of claims 1, or 2, wherein said administering comprises orally administering to said human a liquid or solid preparation containing said pharmaceutical composition.

15. The method according to any of claims 1, or 2, wherein said administering comprises topically applying to skin surface of said human said pharmaceutical composition.

16. The method according to any of claims 1, or 2, wherein said pharmaceutical composition is selected from the group consisting of a pellet, a tablet, a capsule, a solution, a suspension, an emulsion, an elixir, a gel, a cream, and a suppository.

17. The method according to claim 16, wherein said suppository is a rectal suppository or a urethral suppository.

18. The method according to any of claims 1, or 2, wherein said pharmaceutical composition is a parenteral formulation.

19. The method according to claim 18, wherein said parenteral formulation comprises a liposome.

20. The method according to any of claims 1, or 2, wherein said pharmaceutical composition is administered once daily.

21. The method according to any of claims 1, or 2, wherein said pharmaceutical composition is administered twice daily.

22. The method according to any of claims 1, or 2, wherein said pharmaceutical composition is administered thrice daily.
